(19) United States
(12) Patent Application Publication
(10) Pub. No.: US 2006/0020015 A1
(43) Pub. Date: Jan. 26, 2006

(54) Method for treating nervous system disorders and conditions

(75) Inventors: Magid A. Abou-Gharbia, Exton, PA (US); Arthur Jay Cohn, Lafayette Hill, PA (US); Darlene Coleman Deecher, Quakertown, PA (US)

Correspondence Address: WOODCOCK WASHBURN LLP, ONE LIBERTY PLACE - 46TH FLOOR, PHILADELPHIA, PA, 19103, US

(73) Assignee: Wyeth, Madison, NJ (US)

(21) Appl. No.: 11/186,279

(22) Filed: Jul. 21, 2005

Related U.S. Application Data
(60) Provisional application No. 60/590,203, filed on Jul. 22, 2004.

(51) Int. Cl. A61K 31/403

(52) U.S. Cl. 514/412

(57) ABSTRACT

The present invention is directed to racemic 1-(3,4-dichlorophenyl)-3-azabicyclo[3.1.0]hexane, (+)-1-(3,4-dichlorophenyl)-3-azabicyclo[3.1.0]hexane, racemic 1-(4-methylphenyl)-3-azabicyclo[3.1.0]hexane, and (+)-1-(4-methylphenyl)-3-azabicyclo[3.1.0]hexane, and methods of their use for treating certain nervous system disorders and conditions, including, inter alia, vasomotor symptoms (VMS) and chronic pain.

### CROSS REFERENCE TO RELATED APPLICATION

This application claims the benefit of U.S. Application No. 60/590,203 filed Jul. 22, 2004, the entire disclosure of which is incorporated herein by reference.

### FIELD OF THE INVENTION

The present invention relates to racemic 1-(3,4-dichlorophenyl)-3-azabicyclo[3.1.0]hexane, (+)-1-(3,4-dichlorophenyl)-3-azabicyclo[3.1.0]hexane, racemic 1-(4-methylphenyl)-3-azabicyclo[3.1.0]hexane, and (+)-1-(4-methylphenyl)-3-azabicyclo[3.1.0]hexane, and methods of their use for treating certain nervous system disorders and conditions, including, inter alia, vasomotor symptoms (VMS) and chronic pain.

### BACKGROUND OF THE INVENTION

Vasomotor symptoms (VMS), referred to as hot flushes and night sweats, are the most common symptoms associated with menopause, occurring in 60% to 80% of all women following natural or surgically-induced menopause. VMS are likely to be an adaptive response of the central nervous system (CNS) to declining sex steroids. To date, the most effective therapies for VMS are hormone-based treatments, including estrogens and/or some progestins. Hormonal treatments are very effective at alleviating VMS, but they are not appropriate for all women. It is well recognized that VMS are caused by fluctuations of sex steroid levels and can be disruptive and disabling in both males and females. A hot flush can last up to thirty minutes and vary in its frequency from several times a week to multiple occurrences per day. The patient experiences a hot flush as a sudden feeling of heat that spreads quickly from the face to the chest and back and then over the rest of the body. It is usually accompanied by outbreaks of profuse sweating. It may sometimes occur several times an hour, and it often occurs at night. Hot flushes and outbreaks of sweats occurring during the night can cause sleep deprivation. Psychological and emotional symptoms observed, such as nervousness, fatigue, irritability, insomnia, depression, memory loss, headache, anxiety, nervousness or inability to concentrate are considered to be caused by the sleep deprivation following hot flush and night sweats (Kramer et al., In: Murphy et al., 3^{rd} Int'l Symposium on Recent Advances in Urological Cancer Diagnosis and Treatment-Proceedings, Paris, France: SCI: 3-7 (1992)).

Hot flushes may be even more severe in women treated for breast cancer for several reasons: (1) many survivors of breast cancer are given tamoxifen, the most prevalent side effect of which is hot flush; (2) many women treated for breast cancer undergo premature menopause from chemotherapy; (3) women with a history of breast cancer have generally been denied estrogen therapy because of concerns about potential recurrence of breast cancer (Loprinzi, et al., Lancet, 2000, 356(9247): 2059-2063).

Men also experience hot flushes following steroid hormone (androgen) withdrawal. This is true in cases of age-associated androgen decline (Katovich, et al., Proceedings of the Society for Experimental Biology & Medicine, 1990, 193(2): 129-35) as well as in extreme cases of hormone deprivation associated with treatments for prostate cancer (Berendsen, et al., European Journal of Pharmacology, 2001, 419(1): 47-54). As many as one-third of these patients will experience persistent and frequent symptoms severe enough to cause significant discomfort and inconvenience.

The precise mechanism of the VMS is unknown but generally is thought to represent disturbances to normal homeostatic mechanisms controlling thermoregulation and vasomotor activity (Kronenberg et al., “Thermoregulatory Physiology of Menopausal Hot Flashes: A Review,” Can. J. Physiol. Pharmacol., 1987, 65:1312-1324).

The fact that estrogen treatment (e.g. estrogen replacement therapy) relieves the symptoms establishes the link between these symptoms and an estrogen deficiency. For example, the menopausal stage of life is associated with a wide range of other acute symptoms, as described above, and these symptoms are generally estrogen responsive.

It has been suggested that estrogens may stimulate the activity of both the norepinephrine (NE) and/or serotonin (5-HT) systems (J. Pharmacology & Experimental Therapeutics, 1986, 236(3) 646-652). It is hypothesized that estrogens modulate NE and 5-HT levels providing homeostasis in the thermoregulatory center of the hypothalamus. The descending pathways from the hypothalamus via brainstem/spinal cord and the adrenals to the skin are involved in maintaining normal skin temperature. The action of NE and 5-HT reuptake inhibitors is known to impinge on both the CNS and peripheral nervous system (PNS). The pathophysiology of VMS is mediated by both central and peripheral mechanisms and, therefore, the interplay between the CNS and PNS may account for the efficacy of dual acting SRI/NRIs in the treatment of thermoregulatory dysfunction. In fact, the physiological aspects and the CNS/PNS involvement in VMS may account for the lower doses proposed to treat VMS (Loprinzi, et al., Lancet, 2000, 356:2059-2063; Stearns et al., JAMA, 2003, 289:2827-2834) compared to doses used to treat the behavioral aspects of depression. The interplay of the CNS/PNS in the pathophysiology of VMS and the presented data within this document were used to support the claims that the norepinephrine system could be targeted to treat VMS.

Although patients with VMS are most commonly treated by hormone therapy (orally, transdermally, or via an implant), some patients cannot tolerate estrogen treatment (Berendsen, Maturitas, 2000, 36(3): 155-164, Fink et al., Nature, 1996, 383(6598): 306). In addition, hormone replacement therapy is usually not recommended for women or men with or at risk for hormonally sensitive cancers (e.g. breast or prostate cancer). Thus, non-hormonal therapies (e.g. fluoxetine, paroxetine [SRIs] and clonidine) are being evaluated clinically. WO9944601 discloses a method for decreasing hot flushes in a human female by administering fluoxetine. Other options have been studied for the treatment of hot flushes, including steroids, alpha-adrenergic agonists, and beta-blockers, with varying degree of success (Waldinger et al., Maturitas, 2000, 36(3): 165-168).

It has been reported that α₂-adrenergic receptors play a role in thermoregulatory dysfunctions (Freedman et al., Fertility & Sterility, 2000, 74(1): 20-3). These receptors are located both pre- and post-synaptically and mediate an inhibitory role in the central and peripheral nervous system. There are four distinct subtypes of the adrenergic_{α2} receptors, i.e., are α_{2A}, α_{2B}, α_{2C} and α_{2D} (Mackinnon et al., TIPS, 1994, 15: 119; French, Pharmacol. Ther., 1995, 68: 175). It has been reported that a non-select α₂-adrenoceptor antagonist, yohimbine, induces a flush and an α₂-adrenergic receptor agonist, clonidine, alleviates the yohimbine effect (Katovich, et al., Proceedings of the Society for Experimental Biology & Medicine, 1990, 193(2): 129-35, Freedman et al., Fertility & Sterility, 2000, 74(1): 20-3). Clonidine has been used to treat hot flush. However, using such treatment is associated with a number of undesired side effects caused by high doses necessary to abate hot flush described herein and known in the related arts.

Given the complex multifaceted nature of thermoregulation and the interplay between the CNS and PNS in maintaining thermoregulatory homeostasis, multiple therapies and approaches can be developed to target vasomotor symptoms. The present invention focuses on methods directed to these and other important uses for treating nervous system disorders and conditions.

### SUMMARY OF THE INVENTION

The present invention is directed to racemic 1-(3,4-dichlorophenyl)-3-azabicyclo[3.1.0]hexane, (+)-1-(3,4-dichlorophenyl)-3-azabicyclo[3.1.0]hexane, racemic 1-(4-methylphenyl)-3-azabicyclo[3.1.0]hexane, and (+)-1-(4-methylphenyl)-3-azabicyclo[3.1.0]hexane, all of which are norepinephrine reuptake inhibitors (NRI), and methods of their use for treating nervous system disorders or conditions, including, inter alia, vasomotor symptoms (VMS) and chronic pain.

In one embodiment, the present invention is directed to methods for treating at least one nervous system disorder or condition in a subject in need thereof, comprising the step of:
administering to said subject a composition comprising an effective amount of racemic 1-(3,4-dichlorophenyl)-3-azabicyclo[3.1.0]hexane, racemic 1-(4-methylphenyl)-3-azabicyclo[3.1.0]hexane, or a pharmaceutically acceptable salt thereof;
wherein said nervous system disorder or condition is a vasomotor symptom, sexual arousal and desire, fibromyalgia, chronic fatigue, hypothalamic amenorrhea, chronic pain, cognitive dysfunction associated with senile dementia, memory loss, Alzheimer's disease, amnesia, autism, Shy Drager syndrome, Raynaud's syndrome and pain associated therewith, epilepsy, Lennox syndrome, intellectual deficit associated with cerebrovascular disease, schizophrenia, schizoaffective disorder, schizophreniform disorder, seasonal affective disorder, sleep disorder, premenstrual dysphoric disorder, withdrawal syndrome, bipolar disorder, cyclothymic disorder, dysthymic disorder, generalized anxiety disorder, social phobia, selective serotonin reuptake inhibition (SSRI) poop out syndrome, panic disorder, agoraphobia, post traumatic stress disorder, borderline personality disorder, fecal incontinence, disturbances of consciousness, coma, speech disorders, or a combination thereof.

In another embodiment, the present invention is directed to methods for treating at least one nervous system disorder or condition in a subject in need thereof, comprising the step of:
administering to said subject a composition comprising an effective amount of (+)-1-(3,4-dichlorophenyl)-3-azabicyclo[3.1.0]hexane, (+)-1-(4-methylphenyl)-3-azabicyclo[3.1.0]hexane, or a pharmaceutically acceptable salt thereof;
wherein said nervous system disorder or condition is a vasomotor symptom, sexual arousal and desire, fibromyalgia, chronic fatigue, hypothalamic amenorrhea, chronic pain, cognitive dysfunction associated with senile dementia, memory loss, Alzheimer's disease, amnesia, autism, Shy Drager syndrome, Raynaud's syndrome and pain associated therewith, epilepsy, Lennox syndrome, intellectual deficit associated with cerebrovascular disease, schizophrenia, schizoaffective disorder, schizophreniform disorder, seasonal affective disorder, sleep disorder, premenstrual dysphoric disorder, withdrawal syndrome, attention-deficit disorder with or without hyperactivity disorder, bipolar disorder, cyclothymic disorder, dysthymic disorder, generalized anxiety disorder, social phobia, selective serotonin reuptake inhibition (SSRI) poop out syndrome, panic disorder, agoraphobia, post traumatic stress disorder, Gilles de la Tourette Syndrome, borderline personality disorder, fecal incontinence, disturbances of consciousness, coma, speech disorders, hyperkinetic syndrome, or a combination thereof.

### BRIEF DESCRIPTION OF THE DRAWINGS

The invention can be more fully understood from the following detailed description and the accompanying drawings that form a part of this application.

FIG. 1 is an overview of estrogen action on norepinephrine/serotonin mediated thermoregulation.

FIG. 2 is a schematic representation of the interactions of norepinephrine and serotonin and their respective receptors (5-HT₂ₐ, α₁ and α₂-adrenergic).

FIG. 3 is a plot of % uptake as a function of concentration for the norepinephrine (NE) uptake assay, serotonin (5-HT) uptake assay, and dopamine transporter (hDAT) membrane binding assay for racemic 1-(3,4-dichlorophenyl)-3-azabicyclo[3.1.0]hexane (referred to in EXAMPLE 1).

FIGS. 4, 5, and 6 show the results of the administration of racemic-1-(3,4-dichlorophenyl)-3-azabicyclo[3.1.0]hexane, racemic 1-(4-methylphenyl)-3-azabicyclo[3.1.0]hexane (bicifadine), and (+)-1-(4-methylphenyl)-3-azabicyclo[3.1.0]hexane at 1 dose (30 mg/kg, sc) in telemetry rat model of ovariectomy-induced thermoregulatory dysfunction (referred to in EXAMPLE 2).

FIG. 7 is a plot of 50% threshold sensitivity values (50% threshold in grams force) estimated by the Dixon non-parametric test at pre-operative (Pre), baseline (BL), and 30, 60, 100, 180, and 300 minutes after administration of racemic 1-(3,4-dichlorophenyl)-3-azabicyclo[3.1.0]hexane and vehicle (referred to in EXAMPLE 3).

FIG. 8 is a plot of % reversal at 30, 60, 100, 180, and 300 minutes after administration of racemic 1-(4-methylphenyl)-3-azabicyclo[3.1.0]hexane (bicifadine), (+)-1-(4-methylphenyl)-3-azabicyclo[3.1.0]hexane, (−)-1-(4-methylphenyl)-3-azabicyclo[3.1.0]hexane, gabapentin, and vehicle (referred to in EXAMPLE 3).

### DETAILED DESCRIPTION OF THE INVENTION

The present invention is directed to racemic 1-(3,4-dichlorophenyl)-3-azabicyclo[3.1.0]hexane, (+)-1-(3,4-dichlorophenyl)-3-azabicyclo[3.1.0]hexane, racemic 1-(4-methylphenyl)-3-azabicyclo[3.1.0]hexane (also known as bicifadine), and (+)-1-(4-methylphenyl)-3-azabicyclo[3.1.0]hexane (also known as (1S,5R)-1-((4-methylphenyl)-3-azabicyclo[3.1.0]hexane), all of which are norepinephrine reuptake inhibitors (NRI), and methods of their use for treating nervous system disorders and conditions, including, inter alia, vasomotor symptoms (VMS) and chronic pain.

The following definitions are provided for the full understanding of terms and abbreviations used in this specification.

As used herein and in the appended claims, the singular forms “a,” “an,” and “the” include the plural reference unless the context clearly indicates otherwise. Thus, for example, a reference to “an antagonist” includes a plurality of such antagonists, and a reference to “a compound” is a reference to one or more compounds and equivalents thereof known to those skilled in the art, and so forth.

The abbreviations in the specification correspond to units of measure, techniques, properties, or compounds as follows: “min” means minutes, “h” means hour(s), “μL” means microliter(s), “mL” means milliliter(s), “mM” means millimolar, “M” means molar, “mmole” means millimole(s), “cm” means centimeters, “SEM” means standard error of the mean and “IU” means International Units. “ED₅₀ value” means dose which results in 50% alleviation of the observed condition or effect (50% mean maximum endpoint). Optical rotations are measured for compounds in their HCl salt form, unless otherwise noted.
“Norepinephrine transporter” is abbreviated NET.
“Human norepinephrine transporter” is abbreviated hNET.
“Serotonin transporter” is abbreviated SERT.
“Human serotonin transporter” is abbreviated hSERT.
“Norepinephrine reuptake inhibitor” is abbreviated NRI.
“Selective norepinephrine reuptake inhibitor” is abbreviated SNRI.
“Serotonin reuptake inhibitor” is abbreviated SRI.
“Selective serotonin reuptake inhibitor” is abbreviated SSRI.
“Norepinephrine” is abbreviated NE.
“Serotonin is abbreviated 5-HT.
“Subcutaneous” is abbreviated sc.
“Intraperitoneal” is abbreviated ip.
“Oral” is abbreviated po.

As used herein, the term “treatment” includes preventative (e.g., prophylactic), curative or palliative treatment and “treating” as used herein also includes preventative, curative and palliative treatment.

As used herein, the term “effective amount” refers to an amount effective, at dosages, and for periods of time necessary, to achieve the desired result with respect to the treatment of the nervous system disorder or condition. In particular, with respect to vasomotor symptoms, “effective amount” refers to the amount of compound or composition of compounds that would increase norepinephrine levels to compensate in part or total for the lack of steroid availability in subjects afflicted with a vasomotor symptom. Varying hormone levels will influence the amount of compound required in the present invention. For example, the pre-menopausal state may require a lower level of compound due to higher hormone levels than the peri-menopausal state.

It will be appreciated that the effective amount of components of the present invention will vary from patient to patient not only with the particular compound, component or composition selected, the route of administration, and the ability of the components (alone or in combination with one or more combination drugs) to elicit a desired response in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage ultimately being at the discretion of the attendant physician. Dosage regimens may be adjusted to provide the improved therapeutic response. An effective amount is also one in which any toxic or detrimental effects of the components are outweighed by the therapeutically beneficial effects.

Preferably, the compounds useful in the methods of the present invention are administered at a dosage and for a time such that the number of VMS, particularly hot flush, is reduced as compared to the number of VMS before the start of treatment. Such treatment can also be beneficial to reduce the overall severity or intensity distribution of any VMS, especially, hot flushes still experienced, as compared to the severity of the VMS before the start of the treatment. With respect to the other nervous system disorders or condition, including chronic pain, the compounds useful in the methods of the present invention are administered at a dosage and for a time such that there is the prevention, alleviation, or elimination of the symptom of the disorder or condition.

For example, for an afflicted patient, racemic 1-(3,4-dichlorophenyl)-3-azabicyclo[3.1.0]hexane, (+)-1-(3,4-dichlorophenyl)-3-azabicyclo[3.1.0]hexane, racemic 1-(4-methylphenyl)-3-azabicyclo[3.1.0]hexane, or (+)-1-(4-methylphenyl)-3-azabicyclo[3.1.0]hexane, may be administered, preferably, at a dosage of from about 0.1 mg/day to about 200 mg/day, more preferably from about 1 mg/day to about 150 mg/day, even more preferably from about 1 mg/day to about 100 mg/day and most preferably from about 1 mg/day to 50 mg/day for a time sufficient to reduce and/or substantially eliminate the nervous system disorder or condition, for example, the number and/or severity of VMS and/or duration and/or severity of chronic pain.

As used herein, the terms “composition of compounds,” “compound,” “drug,” “therapeutic agent,” “pharmacologically active agent,” “active agent,” and “medicament” are used interchangeably herein to refer to a compound or compounds or composition of matter which, when administered to a subject (human or animal) induces a desired pharmacological and/or physiologic effect by local and/or systemic action.

As used herein, the term “modulation” refers to the capacity to either enhance or inhibit a functional property of a biological activity or process, for example, receptor binding or signaling activity. Such enhancement or inhibition may be contingent on the occurrence of a specific event, such as activation of a signal transduction pathway and/or may be manifest only in particular cell types. The modulator is intended to comprise any compound, e.g., antibody, small molecule, peptide, oligopeptide, polypeptide, or protein, preferably small molecule, or peptide.

As used herein, the term “inhibitor” is intended to comprise any compound or agent, e.g., antibody, small molecule, peptide, oligopeptide, polypeptide, or protein, preferably small molecule or peptide, that exhibits a partial, complete, competitive and/or inhibitory effect on mammal by inhibiting, suppressing, repressing, or decreasing a specific activity, such as serotonin reuptake activity or the norepinephrine reuptake activity. In certain embodiments, the term preferably refers to an inhibitor of human norepinephrine reuptake or both serotonin reuptake and norepinephrine reuptake, thus diminishing or blocking, preferably diminishing, some or all of the biological effects of endogenous norepinephrine reuptake or of both serotonin reuptake and the norepinephrine reuptake.

Within the present invention, the racemic 1-(3,4-dichlorophenyl)-3-azabicyclo[3.1.0]hexane, (+)-1-(3,4-dichlorophenyl)-3-azabicyclo[3.1.0]hexane, racemic 1-(4-methylphenyl)-3-azabicyclo[3.1.0]hexane, and (+)-1-(4-methylphenyl)-3-azabicyclo[3.1.0]hexane, may be prepared in the form of pharmaceutically acceptable salts. As used herein, the term “pharmaceutically acceptable salts” refers to salts prepared from pharmaceutically acceptable non-toxic acids, including inorganic salts, and organic salts. Suitable non-organic salts include inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferably is the hydrochloride salt.

As used herein, term “administering” means either directly administering a compound or composition of the present invention, or administering a prodrug, derivative or analog which will form an equivalent amount of the active compound or substance within the body.

As used herein, the term “subject” or “patient” refers to an animal including the human species that is treatable with the compositions, and/or methods of the present invention. The term “subject” or “subjects” is intended to refer to both the male and female gender unless one gender is specifically indicated. Accordingly, the term “patient” comprises any mammal which may benefit from treatment of a nervous system disorder or condition, including, inter alia, vasomotor symptoms and/or chronic pain, such as a human, especially if the mammal is female, either in the pre-menopausal, peri-menopausal, or post-menopausal period. Furthermore, the term patient includes female animals including humans and, among humans, not only women of advanced age who have passed through menopause but also women who have undergone hysterectomy or for some other reason have suppressed estrogen production, such as those who have undergone long-term administration of corticosteroids, suffer from Cushing's syndrome or have gonadal dysgenesis. However, the term “patient” is not intended to be limited to a female.

As used herein, the terms “vasomotor symptoms,” “vasomotor instability symptoms” and “vasomotor disturbances” include, but are not limited to, hot flushes (flashes), insomnia, sleep disturbances, mood disorders, irritability, excessive perspiration, night sweats, fatigue, and the like, caused by, inter alia, thermoregulatory dysfunction.

As used herein, the terms “hot flush” or “hot flash” is an art-recognized term that refers to an episodic disturbance in body temperature typically consisting of a sudden skin flushing, usually accompanied by perspiration in a subject.

As used herein, the terms “premature menopause” or “artificial menopause” refer to ovarian failure of unknown cause that may occur before age 40. It may be associated with smoking, living at high altitude, or poor nutritional status. Artificial menopause may result from oophorectomy, chemotherapy, radiation of the pelvis, or any process that impairs ovarian blood supply.

As used herein, the term “pre-menopausal” means before the menopause, the term “peri-menopausal” means during the menopause and the term “post-menopausal” means after the menopause. “Ovariectomy” means removal of an ovary or ovaries and can be effected according to Merchenthaler, et al., Maturitas, 1998, 30(3): 307-316.

As used herein, the term “chronic pain” refers to centralized or peripheral pain that is intense, localized, sharp, or stinging, and/or dull, aching, diffuse, or burning in nature and that occurs for extended periods of time (i.e., persistent), including, for the purpose of the present invention, neuropathic pain and cancer pain. Chronic pain includes neuropathic pain, hyperalgesia, and/or allodynia.

As used herein, the term “neuropathic pain” refers to chronic pain caused by damage to or pathological changes in the peripheral or central nervous systems. Examples of pathological changes related to neuropathic pain include prolonged peripheral or central neuronal sensitization, central sensitization related damage to nervous system inhibitory and/or exhibitory functions and abnormal interactions between the parasympathetic and sympathetic nervous systems. A wide range of clinical conditions may be associated with or form the basis for neuropathic pain including for example diabetes, post traumatic pain of amputation, lower back pain, cancer, chemical injury, or toxins, other major surgeries, peripheral nerve damage due to traumatic injury compression, nutritional deficiencies, or infections such as shingles or human immunodeficiency virus (HIV). Neuropathic pain may be associated with, for example, diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, or nerve damage caused by injury resulting in peripheral and/or central sensitization such as phantom limb pain, reflex sympathetic dystrophy or postthoracotomy pain, cancer, chemical injury, toxins, nutritional deficiencies, or viral or bacterial infections such as shingles or HIV, or combinations thereof. The methods of use for compounds of this invention further include treatments in which the neuropathic pain is a condition secondary to metastatic infiltration, adiposis dolorosa, burns or central pain conditions related to thalamic conditions, or combinations thereof.

As used herein, the term “hyperalgesia” refers to pain where there is an increase in sensitivity to a typically noxious stimulus.

As used herein, the term “allodynia” refers to an increase in sensitivity to a typically non-noxious stimulus.

As used herein, the term “fibromyalgia” includes, but is not limited to, fibromylagia syndrome (FMS) and other somatoform disorders including FMS associated with depression, somatization disorder, conversion disorder, pain disorder, hypochondriasis, body dysmorphic disorder, undifferentiated somatoform disorder, and somatoform NOS. FMS and other somatoform disorders are accompanied by physiological symptoms selected from a generalized heightened perception of sensory stimuli, abnormalities in pain perception in the form of hyperalgesia, and combinations thereof.

As used herein, the term “chronic fatigue” is a condition associated with physiological symptoms including weakness, muscle aches and pains, excessive sleep, malaise, fever, sore throat, tender lymph nodes, impaired memory and/or mental concentration, insomnia, disordered sleep, localized tenderness, diffuse pain and fatigue, and combinations thereof.

As used herein, the term “sleep disorder” includes, but is not limited to, insomnia, narcolepsy, and enuresis.

As used herein, the term “social phobia” includes, but is not limited to, social anxiety disorder.

As used herein, the term “selective serotonin reuptake inhibition (SSRI) poop out syndrome” refers to a condition where a patient fails to maintain a satisfactory response to SSRI therapy after an initial period of satisfactory response.

As used herein, the term “side effect” refers to a consequence other than the one(s) for which an agent or measure is used, as the adverse effects produced by a drug, especially on a tissue or organ system other then the one sought to be benefited by its administration. In the case, for example, of high doses of NRIs or NRI/SRI compounds alone, the term “side effect” may refer to such conditions as, for example, vomiting, nausea, sweating, and flushes (Janowsky, et al., Journal of Clinical Psychiatry, 1984, 45(10 Pt 2): 3-9).

As used herein, the phrase “substantially free of (−)-1-(3,4-dichlorophenyl)-3-azabicyclo[3.1.0]hexane or a pharmaceutically acceptable salt thereof” means a composition containing no more than about 5% by weight based on the total weight of the composition (w/w) of (−)-1-(3,4-dichlorophenyl)-3-azabicyclo[3.1.0]hexane or a pharmaceutically acceptable salt thereof, preferably less than about 2% w/w, and more preferably less than about 1% w/w.

As used herein, the phrase “substantially free of (−)-1-(4-methylphenyl)-3-azabicyclo[3.1.0]hexane or a pharmaceutically acceptable salt thereof” means a composition containing no more than about 5% by weight based on the total weight of the composition (w/w) of (−)-1-(4-methylphenyl)-3-azabicyclo[3.1.0]hexane (also known as (1S,5R)-1-((4-methylphenyl)-3-azabicyclo[3.1.0]hexane) or a pharmaceutically acceptable salt thereof, preferably less than about 2% w/w, and more preferably less than about 1% w/w.

The present invention is directed to racemic 1-(3,4-dichlorophenyl)-3-azabicyclo[3.1.0]hexane, (+)-1-(3,4-dichlorophenyl)-3-azabicyclo[3.1.0]hexane, racemic 1-(4-methylphenyl)-3-azabicyclo[3.1.0]hexane, and (+)-1-(4-methylphenyl)-3-azabicyclo[3.1.0]hexane (also known as (1R,5S)-1-((4-methylphenyl)-3-azabicyclo[3.1.0]hexane measured as HCl salt), and methods of their use for the treatment of certain nervous disorders and conditions. It is believed that the present invention described presents a substantial breakthrough in the field of treatment, alleviation, inhibition, and/or prevention of nervous system disorders and conditions, including, inter alia, vasomotor symptoms and/or chronic pain.

In one embodiment, the present invention is directed to methods for treating at least one nervous system disorder or condition in a subject in need thereof, comprising the step of:
administering to said subject a composition comprising an effective amount of racemic 1-(3,4-dichlorophenyl)-3-azabicyclo[3.1.0]hexane, racemic 1-(4-methylphenyl)-3-azabicyclo[3.1.0]hexane, or a pharmaceutically acceptable salt thereof;
wherein said nervous system disorder or condition is a vasomotor symptom, sexual arousal and desire, fibromyalgia, chronic fatigue, hypothalamic amenorrhea, chronic pain, cognitive dysfunction associated with senile dementia, memory loss, Alzheimer's disease, amnesia, autism, Shy Drager syndrome, Raynaud's syndrome and pain associated therewith, epilepsy, Lennox syndrome, intellectual deficit associated with cerebrovascular disease, schizophrenia, schizoaffective disorder, schizophreniform disorder, seasonal affective disorder, sleep disorder, premenstrual dysphoric disorder, withdrawal syndrome, bipolar disorder, cyclothymic disorder, dysthymic disorder, generalized anxiety disorder, social phobia, selective serotonin reuptake inhibition (SSRI) poop out syndrome, panic disorder, agoraphobia, post traumatic stress disorder, borderline personality disorder, fecal incontinence, disturbances of consciousness, coma, speech disorders, or a combination thereof. In certain preferred embodiments, the nervous system disorder or condition is a vasomotor symptom, sexual dysfunction, fibromyalgia, chronic fatigue, hypothalamic amenorrhea, chronic pain, cognitive dysfunction associated with senile dementia, memory loss, Alzheimer's disease, amnesia, autism, Shy Drager syndrome, Raynaud's syndrome and pain associated therewith, epilepsy, Lennox syndrome, intellectual deficit associated with cerebrovascular disease, schizophrenia, premenstrual dysphoric disorder, or a combination thereof, especially a vasomotor symptom or chronic pain, especially neuropathic pain, and even more especially neuropathic pain excluding chronic back pain.

In another embodiment, the present invention is directed to methods for treating at least one nervous system disorder or condition in a subject in need thereof, comprising the step of:
administering to said subject a composition comprising an effective amount of (+)-1-(3,4-dichlorophenyl)-3-azabicyclo[3.1.0]hexane, (+)-1-(4-methylphenyl)-3-azabicyclo[3.1.0]hexane, or a pharmaceutically acceptable salt thereof;
wherein said nervous system disorder or condition is a vasomotor symptom, sexual arousal and desire, fibromyalgia, chronic fatigue, hypothalamic amenorrhea, chronic pain, cognitive dysfunction associated with senile dementia, memory loss, Alzheimer's disease, amnesia, autism, Shy Drager syndrome, Raynaud's syndrome and pain associated therewith, epilepsy, Lennox syndrome, intellectual deficit associated with cerebrovascular disease, schizophrenia, schizoaffective disorder, schizophreniform disorder, seasonal affective disorder, sleep disorder, premenstrual dysphoric disorder, withdrawal syndrome, attention-deficit disorder with or without hyperactivity disorder, bipolar disorder, cyclothymic disorder, dysthymic disorder, generalized anxiety disorder, social phobia, selective serotonin reuptake inhibition (SSRI) poop out syndrome, panic disorder, agoraphobia, post traumatic stress disorder, Gilles de la Tourette Syndrome, borderline personality disorder, fecal incontinence, disturbances of consciousness, coma, speech disorders, hyperkinetic syndrome, or a combination thereof.

In certain preferred embodiments, the nervous system disorder or condition is a vasomotor symptom, sexual arousal and desire, fibromyalgia, chronic fatigue, hypothalamic amenorrhea, chronic pain, cognitive dysfunction associated with senile dementia, memory loss, Alzheimer's disease, amnesia, autism, Shy Drager syndrome, Raynaud's syndrome and pain associated therewith, epilepsy, Lennox syndrome, intellectual deficit associated with cerebrovascular disease, schizophrenia, premenstrual dysphoric disorder, or a combination thereof, especially a vasomotor symptom or chronic pain, especially neuropathic pain, and even more especially neuropathic pain excluding chronic back pain. In certain preferred embodiments, composition is substantially free of the corresponding (−)-enantiomer or a pharmaceutically acceptable salt thereof, preferably, comprises less than about 2% w/w of the corresponding (−)-enantiomer or a pharmaceutically acceptable salt thereof, and more preferably, less than about 1% w/w of the corresponding (−)-enantiomer or a pharmaceutically acceptable salt thereof.

In certain embodiments, the present invention is directed to methods for treating at least one nervous system disorder or condition in a subject in need thereof, comprising the step of:
administering to said subject a composition comprising an effective amount of racemic (+)-1-(4-methylphenyl)-3-azabicyclo[3.1.0]hexane or (+)-1-(4-methylphenyl)-3-azabicyclo[3.1.0]hexane, or a pharmaceutically acceptable salt thereof;
wherein said nervous system disorder or condition is chronic pain excluding chronic back pain, especially neuropathic pain excluding chronic back pain.

Accordingly, in one embodiment, the present invention is directed to methods for treating at least one vasomotor symptom in a subject in need thereof, comprising the step of:
administering to said subject a composition comprising an effective amount of racemic 1-(3,4-dichlorophenyl)-3-azabicyclo[3.1.0]hexane, racemic 1-(4-methylphenyl)-3-azabicyclo[3.1.0]hexane, or a pharmaceutically acceptable salt thereof.

In another embodiment, the present invention is directed to methods for treating at least one vasomotor symptom in a subject in need thereof, comprising the step of:
administering to said subject a composition comprising an effective amount of (+)-1-(3,4-dichlorophenyl)-3-azabicyclo[3.1.0]hexane, (+)-1-(4-methylphenyl)-3-azabicyclo[3.1.0]hexane, or a pharmaceutically acceptable salt thereof, In certain preferred embodiments, composition is substantially free of the corresponding (−)-enantiomer or a pharmaceutically acceptable salt thereof, preferably, comprises less than about 2% w/w of the corresponding (−)-enantiomer or a pharmaceutically acceptable salt thereof, and more preferably, less than about 1% w/w of the corresponding (−)-enantiomer or a pharmaceutically acceptable salt thereof.

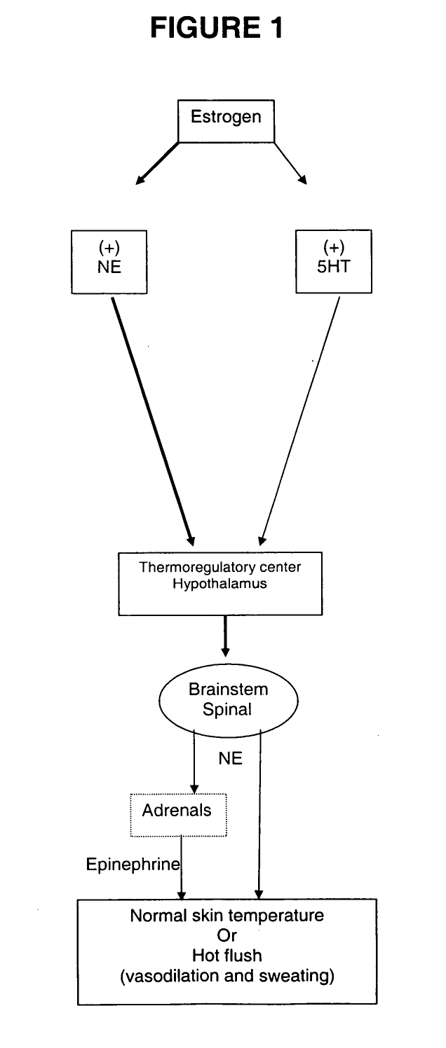

When estrogen levels are low or estrogen is absent, the normal levels between NE and 5-HT is altered and this altered change in neurotransmitter levels may result in changes in the sensitivity of the thermoregulatory center. The altered chemical levels may be translated in the thermoregulatory center as heat sensation and as a response, the hypothalamus may activate the descending autonomic pathways and result in heat dissipation via vasodilation and sweating (hot flush) (FIG. 1). Accordingly, the estrogen deprivation may result in altered norepinephrine activity.

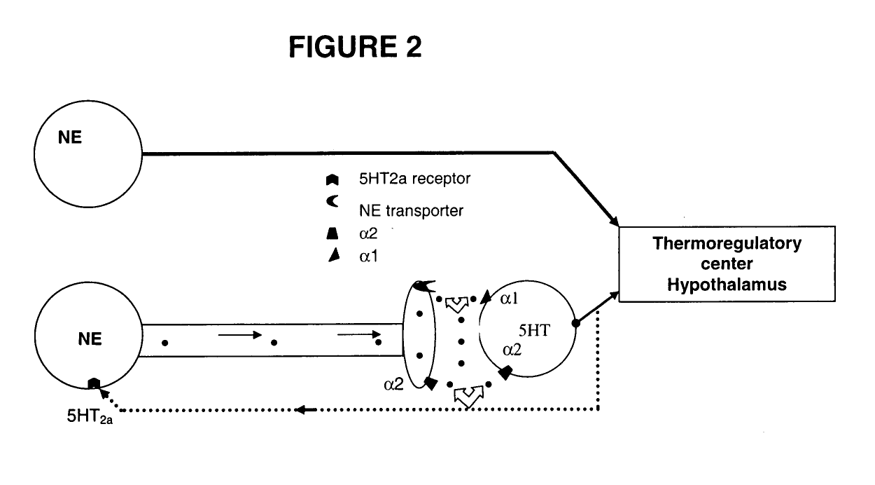

Norepinephrine synthesized in perikarya of the brainstem is released at the nerve terminals in the hypothalamus and brainstem. In the hypothalamus, NE regulates the activity of neurons residing in the thermoregulatory center. In the brainstem, NE innervates serotoninergic neurons (5HT), and acting via adrenergic_{α1} and adrenergic_{α2} postsynaptic receptors, it stimulates the activity of the serotoninergic system. In response, 5-HT neurons also modulate the activity the thermoregulatory center and feedback to NE neurons. Via this feedback connection, 5-HT, acting via 5-HT₂ₐ receptors, inhibits the activity of NE neurons. Norepinephrine in the synaptic cleft is also taken up by NE transporter (NET) located in NE neurons. The transporter recycles NE and makes it available for multiple neurotransmission (FIG. 2).

The present invention provides methods of treating VMS, chronic pain, and/or other nervous system disorders by recovering the reduced activity of norepinephrine, i.e., by inhibiting the reuptake of norepinephrine. Norepinephrine activity in the hypothalamus or in the brainstem can be elevated by (i) blocking the activity of the NE transporter, (ii) blocking the activity of the presynaptic adrenergic_{α2} receptor with an antagonist, or (iii) blocking the activity of 5-HT on NE neurons with a 5-HT₂ₐ antagonist.

In yet other embodiments, the present invention is directed to methods for treating chronic pain, particularly neuropathic pain, in a subject in need thereof, comprising the step of:
administering to said subject a composition comprising an effective amount of racemic 1-(3,4-dichlorophenyl)-3-azabicyclo[3.1.0]hexane, racemic 1-(4-methylphenyl)-3-azabicyclo[3.1.0]hexane, or a pharmaceutically acceptable salt thereof.

Neuropathic pain may be associated with, for example, diabetic neuropathy, post-herpetic neuralgia, trigeminal neuralgia, complex regional pain syndrome, lumbar or cervical radiculopathies, fibromyalgia, glossopharyngeal neuralgia, reflex sympathetic dystrophy, causalgia, thalamic syndrome, nerve root avulsion, monoclonal gammopathy of undetermined significance (MGUS) neuropathy, sarcoid polyneuropathy, HIV-related neuropathy arising from a variety of causes such as from medication used to treat HIV, peripheral neuropathy such as peripheral neuropathy with connective tissue disease, paraneoplastic sensory neuropathy, familial amyloid polyneuropathy, acquired amyloid polyneuropathy, inherited neuropathy, neuropathy with renal failure, hereditary sensory autonomic neuropathy, Fabry's disease, Celiac disease or nerve damage cause by injury resulting in peripheral and/or central sensitization such as phantom limb pain, reflex sympathetic dystrophy or postthoracotomy pain, cancer including neuropathies caused by chemotherapy agents or other agents used to treat the disease, chemical injury, toxins such as arsenic neuropathy, nutritional deficiencies, or viral or bacterial infections such as shingles or HIV-related neuropathy, or combinations thereof. The methods of use for compounds of this invention further include treatments in which the neuropathic pain is a condition secondary to metastatic infiltration, adiposis dolorosa, burns, or central pain conditions related to thalamic conditions.

Neuropathic pains described above may also be, in some circumstances, classified as “painful small fiber neuropathies” such as idiopathic small-fiber painful sensory neuropathy, or “painful large fiber neuropathies” such as demylinating neuropathy or axonal neuropathy, or combinations thereof. Such neuropathies are described in more detail, for example, in the J. Mendell et al., N. Engl. J. Med. 2003, 348:1243-1255, which is hereby incorporated by reference in its entirety.

In further embodiments, the present invention is directed to methods for treating chronic pain, particularly neuropathic pain, in a subject in need thereof, comprising the step of:
administering to said subject a composition comprising an effective amount of (+)-1-(3,4-dichlorophenyl)-3-azabicyclo[3.1.0]hexane, (+)-1-(4-methylphenyl)-3-azabicyclo[3.1.0]hexane, or a pharmaceutically acceptable salt thereof.

In certain preferred embodiments, composition is substantially free of the corresponding (−)-enantiomer or a pharmaceutically acceptable salt thereof, preferably, comprises less than about 2% w/w of the corresponding (−)-enantiomer or a pharmaceutically acceptable salt thereof, and more preferably, less than about 1% w/w of the corresponding (−)-enantiomer or a pharmaceutically acceptable salt thereof.

In yet other preferred embodiments, the invention is directed to methods wherein the composition further comprises a therapeutically effective amount of at least one adrenergic_{α2} receptor antagonist or a pharmaceutically acceptable salt thereof. In certain preferred embodiments, the norepinephrine reuptake inhibitor and the adrenergic_{α2} receptor antagonist are administered simultaneously or concurrently. In certain preferred embodiments, the adrenergic_{α2} receptor antagonist is selective for the adrenergic_{α2A} receptor, adrenergic_{α2B} receptor, adrenergic_{α2C} receptor, or adrenergic_{α2D} receptor.

Adrenergic_{α2} receptor antagonists are known to induce hot flush. However, an adrenergic_{α2} receptor antagonist may be co-administered with an NRI compound, to abate hot flush. The dose level may require adjustment according to the dose of adrenergic_{α2} receptor antagonist administered, in order to block side effects without altering the efficacy on hot flushes. One of ordinary skill in the art will know how to determine such doses without undue experimentation.

Examples of adrenergic_{α2} receptor antagonist include, but are not limited to, atipamezole; 2-[2-(4-(2-methoxyphenyl)piperazin-1-yl)ethyl]-4,4-dimethyl-1,3-(2H, 4H)-isoquinolindione dihydrochloride (ARC 239 dihydrochloride); 2-[(4,5-dihydro-1H-imidazol-2-yl)methyl]-2,3-dihydro-1-methyl-1H-isoindole maleate (BRL 44408 maleate); BRL48962; BRL41992; SKF 104856; SKF 104078; MK912; 2-(2-ethyl-2,3-dihydro-2-benzofuranyl)-4,5-dihydro-1H-imidazole hydrochloride (efaroxan hydrochloride); 2-(1,4-benzodioxan-2-yl)-2-imidazoline hydrochloride (idazoxan hydrochloride); 2-(1-ethyl-2-indazoyl)methyl-1,4-benzodioxan hydrochloride (imiloxan hydrochloride); 17α-hydroxy-20α-yohimban-16β-carboxylic acid, methyl ester hydrochloride (rauwolscine hydrochloride); (8αR, 12αS, 13αS)-5,8,8α,9,10,11,12,12α, 13,13α-dechydro-3-methoxy-12-(ethylsulfonyl)-6H-isoquino [2,1-γ][1,6]naphthyridine hydrochloride (RS 79948 hydrochloride); 2-(2,3-dihydro-2-methoxy-1,4-benzodioxin-2-yl)-4,5-dihydro-1H-imidazole hydrochloride (RX 821002 hydrochloride); 8-[(2,3-dihydro-1,4-benzodioxin-2-yl)methyl]-1-phenyl-1,3,8-triaza spiro[4,5]decan-4-one (spiroxatrine); 17α-hydroxyyohimban-16α-carboxylic acid methyl ester hydrochloride (yohimbine hydrochloride); and combinations and pharmaceutically acceptable salts thereof. Several of these compounds are available from Tocris Cookson Inc., Ellisville, Mo.

In certain preferred embodiments, the adrenergic_{α2} receptor antagonist is selective for the adrenergic_{α2A} receptor, adrenergic_{α2B} receptor, adrenergic_{α2C} receptor, or adrenergic_{α2D} receptor. BRL44408 and BRL48962 are known to be selective adrenergic_{α2A} receptor antagonists. Imiloxan is a known selective adrenergic_{α2B} receptor antagonist. Rauwolscine and MK912 are known selective adrenergic_{α2C} receptor antagonists.

The present invention includes prodrugs of the compounds of formula I. As used herein, the term “prodrug” means a compound which is convertible in vivo by metabolic means (e.g. by hydrolysis) to a compound of formula I. Various forms of prodrugs are known in the art, for example, as discussed in Bundgaard, (ed.), Design of Prodrugs, Elsevier (1985); -Widder, et al. (ed.), Methods in Enzymology, vol. 4, Academic Press (1985); Krogsgaard-Larsen, et al., (ed). “Design and Application of Prodrugs,” Textbook of Drug Design and Development, Chapter 5, 113-191 (1991), Bundgaard, et al., Journal of Drug Deliver Reviews, 1992, 8:1-38, Bundgaard, J. of Pharmaceutical Sciences, 1988, 77:285 et seq.; and Higuchi and Stella (eds.) Prodrugs as Novel Drug Delivery Systems, American Chemical Society (1975).

Further, the racemic 1-(3,4-dichlorophenyl)-3-azabicyclo[3.1.0]hexane, (+)-1-(3,4-dichlorophenyl)-3-azabicyclo[3.1.0]hexane, racemic 1-(4-methylphenyl)-3-azabicyclo[3.1.0]hexane, and (+)-1-(4-methylphenyl)-3-azabicyclo[3.1.0]hexane may exist in unsolvated as well as in solvated forms with pharmaceutically acceptable solvents such as water, ethanol, and the like. In general, the solvated forms are considered equivalent to the unsolvated forms for the purpose of the present invention.

The compounds useful in the methods of the present invention may be prepared in a number of ways well known to those skilled in the art. The compounds can be synthesized, for example, by the methods as described below, or variations thereon as appreciated by the skilled artisan. The reagents used in the preparation of the compounds of this invention can be either commercially obtained or can be prepared by standard procedures described in the literature. All processes disclosed in association with the present invention are contemplated to be practiced on any scale, including milligram, gram, multigram, kilogram, multikilogram or commercial industrial scale.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Protecting groups that may be employed in accordance with the present invention may be described in Greene, T. W. and Wuts, P. G. M., Protective Groups in Organic Synthesis 2d. Ed., Wiley & Sons, 1991.

The racemic 1-(3,4-dichlorophenyl)-3-azabicyclo[3.1.0]hexane, (+)-1-(3,4-dichlorophenyl)-3-azabicyclo[3.1.0]hexane, racemic 1-(4-methylphenyl)-3-azabicyclo[3.1.0]hexane, and (+)-1-(4-methylphenyl)-3-azabicyclo[3.1.0]hexane, or a pharmaceutically acceptable salt thereof, may be prepared as described, for example, in U.S. Pat. No. 4,131,611, U.S. Pat. No. 4,435,419, US-B-6,204,284, and US-B-6,372,919, the disclosures of which are incorporated herein by reference.

The (+) enantiomer useful in the method of the invention may be isolated from its racemic mixture by any method known to those skilled in the art, including high performance liquid chromatography (HPLC) and the formation and crystallization of chiral salts or prepared by methods described herein. See, for example, US-B-6,372,912; Jacques, et al., Enantiomers, Racemates and Resolutions (Wiley Interscience, New York, 1981); Wilen, S. H., et al., Tetrahedron, 33:2725 (1977); Eliel, E. L. Stereochemistry of Carbon Compounds, (McGraw-Hill, NY, 1962); Wilen, S. H. Tables of Resolving Agents and Optical Resolutions, p. 268 (E. L. Eliel, Ed., University of Notre Dame Press, Notre Dame, Ind. 1972).

In some embodiments, (+)-enantiomer is obtained by resolving the corresponding racemic mixture using a chiral polysaccharide stationary phase and an organic eluent. Preferably, the polysaccharide is starch or starch derivative. A chiral HPLC column may be used, such as, for example, a CHIRALPAK™ AD HPLC column manufactured by Diacel and commercially available from Chiral Technologies, Inc., Exton, Pa., more preferably a 1 cm×25 cm CHIRALPAK™ AD HPLC column. The preferred eluent is a hydrocarbon solvent adjusted in polarity with a miscible polar organic solvent. Preferably, the organic eluent contains a non-polar, hydrocarbon solvent present in about 95% to about 99.5% (volume/volume) and a polar organic solvent present in about 5% to about 0.5% (volume/volume). In a preferred embodiment, the hydrocarbon solvent is hexane and the miscible polar organic solvent is isopropylamine.

The racemic 1-(3,4-dichlorophenyl)-3-azabicyclo[3.1.0]hexane, (+)-1-(3,4-dichlorophenyl)-3-azabicyclo[3.1.0]hexane, racemic 1-(4-methylphenyl)-3-azabicyclo[3.1.0]hexane, and (+)-1-(4-methylphenyl)-3-azabicyclo[3.1.0]hexane, or pharmaceutically acceptable salts thereof, useful in the methods of the invention may be used as a neat composition or as a composition containing at least one pharmaceutically acceptable carrier. Generally, the azabicyclohexane or a pharmaceutically acceptable salt thereof, will be present at a level of from about 0.1%, by weight, to about 90% by weight, based on the total weight of the composition, based on the total weight of the composition. Preferably, the azabicyclohexane or a pharmaceutically acceptable salt thereof will be present at a level of at least about 1%, by weight, based on the total weight of the composition. More preferably, the azabicyclohexane or a pharmaceutically acceptable salt thereof will be present at a level of at least about 5%, by weight, based on the total weight of the composition. Even more preferably, the azabicyclohexane or a pharmaceutically acceptable salt thereof will be present at a level of at least about 10%, by weight, based on the total weight of the composition. Yet even more preferably, the azabicyclohexane or a pharmaceutically acceptable salt thereof, will be present at a level of at least about 25%, by weight, based on the total weight of the composition.

Such compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable.

The compounds of this invention may be administered orally or parenterally, neat or in combination with conventional pharmaceutical carriers. Applicable solid carriers can include one or more substances that may also act as flavoring agents, lubricants, solubilizers, suspending agents, fillers, glidants, compression aids, binders or tablet-disintegrating agents or an encapsulating material. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes, and ion exchange resins.

Liquid carriers may be used in preparing solutions, suspensions, emulsions, syrups, and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration.

Liquid pharmaceutical compositions, which are sterile solutions or suspensions, can be administered by, for example, intramuscular, intraperitoneal or subcutaneous injection. Sterile solutions can also be administered intravenously. Oral administration may be either liquid or solid composition form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

In another embodiment of the present invention, the compounds useful in the methods of the present invention may be administered to a mammal with one or more other pharmaceutical active agents such as those agents being used to treat any other medical condition present in the mammal. Examples of such pharmaceutical active agents include pain relieving agents, anti-angiogenic agents, anti-neoplastic agents, anti-diabetic agents, anti-infective agents, or gastrointestinal agents, or combinations thereof.

The one or more other pharmaceutical active agents may be administered in a therapeutically effective amount simultaneously (such as individually at the same time, or together in a pharmaceutical composition), and/or successively with one or more compounds of the present invention.

The term “combination therapy” refers to the administration of two or more therapeutic agents or compounds to treat a therapeutic disorder or condition described in the present disclosure, for example hot flush, sweating, thermoregulatory-related condition or disorder, or other. Such administration includes use of each type of therapeutic agent in a concurrent manner. In either case, the treatment regimen will provide beneficial effects of the drug combination in treating the conditions or disorders described herein.

The route of administration may be any route, which effectively transports the active azabicyclohexane compound, or a pharmaceutically acceptable salt thereof, to the appropriate or desired site of action, such as oral, nasal, pulmonary, transdermal, such as passive or iontophoretic delivery, or parenteral, e.g. rectal, depot, subcutaneous, intravenous, intraurethral, intramuscular, intranasal, ophthalmic solution or an ointment. Furthermore, the administration of the azabicyclohexane or pharmaceutically acceptable salt thereof with other active ingredients may be concurrent or simultaneous.

The present invention is further defined in the following Examples, in which all parts and percentages are by weight and degrees are Celsius, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### EXAMPLES

### Example 1

### Activity of racemic 1-(3,4-dichlorophenyl)-3-azabicyclo[3.1.0]hexane and (+)-1-(4-methylphenyl)-3-azabicyclo[3.1.0]hexane at the human norepinephrine (hNET) serotonin (hSERT) and dopamine (hDAT) transporters

Cell Lines and Reagents

MDCK-Net6 cells, stably transfected with human hNET [15] were cultured in growth medium containing high glucose DMEM (Gibco, Cat. No. 11995), 10% FBS (dialyzed, heat-inactivated, US Bio-Technologies, Lot FBD1129HI) and 500 μg/ml G418 (Gibco, Cat. No. 10131). Cells were plated at 300,000/T75 flask and cells were split twice weekly. The JAR cell line (human placental choriocarcinoma) was purchased from ATCC (Cat. No. HTB-144). The cells were cultured in growth medium containing RPMI 1640 (Gibco, Cat. No. 72400), 10% FBS (Irvine, Cat. No. 3000), 1% sodium pyruvate (Gibco, Cat. No. 1136) and 0.25% glucose. Cells were plated at 250,000 cells/T75 flask and split twice weekly. For cell based assays, cells were plated in Wallac 96-well sterile plates (Perkin Elmer, Cat. No. 3983498). For the human dopamine transporter (hDAT) binding assay, membranes from cells expressing recombinant hDAT are purchased from Perkin Elmer (Cat. No. RBHDA™, Lot#2227) and maintained at −80° C. until assay day.

Norepinephrine (NE) Uptake Assay

On day 1, cells were plated at 3,000 cells/well in growth medium and maintained in a cell incubator (37° C., 5% CO₂). On day 2, growth medium was replaced with 200 μl of assay buffer (25 mM HEPES; 120 mM NaCl; 5 mM KCl; 2.5 mM CaCl₂; 1.2 mM MgSO₄; 2 mg/ml glucose (pH 7.4, 37° C.)) containing 0.2 mg/ml ascorbic acid and 10 μM pargyline. Plates containing cells with 200 μl of assay buffer were equilibrated for 10 minutes at 37° C. prior to addition of compounds. A stock solution of desipramine was prepared in DMSO (10 mM) and delivered to triplicate wells containing cells for a final test concentration of 1 μM. Data from these wells were used to define non-specific NE uptake (minimum NE uptake). Test compounds were prepared in DMSO (10 mM) and diluted in assay buffer according to test range (1 to 10,000 nM). Twenty-five microliters of assay buffer (maximum NE uptake) or test compound were added directly to triplicate wells containing cells in 200 μl of assay buffer. The cells in assay buffer with test compounds, were incubated for 20 minutes at 37° C. To initiate the NE uptake, [³H]NE diluted in assay buffer (120 nM final assay concentration) was delivered in 25 μl aliquots to each well and the plates were incubated for 5 minutes (37° C.). The reactions were terminated by decanting the supernatant from the plate. The plates containing cells were washed twice with 200 μl assay buffer (37° C.) to remove free radioligand. The plates were then inverted, left to dry for 2 minutes, then reinverted and air dried for an additional 10 minutes. The cells were lysed in 25 μl of 0.25 N NaOH solution (4° C.), placed on a shake table and vigorously shaken for 5 minutes. After cell lysis, 75 μl of scintillation cocktail was added to each well and the plates were sealed with film tape. The plates were returned to the shake table and vigorously shaken for a minimum of 10 minutes to ensure adequate partitioning of organic and aqueous solutions. The plates were counted in a Wallac Microbeta counter (PerkinElmer) to collect the raw cpm data.

Serotonin (5-HT) Uptake Assay

The methods for 5-HT functional reuptake using the JAR cell line were modified using a previous literature report. On day 1, cells were plated at 15,000 cells/well in 96-well plates containing growth medium (RPMI 1640 with 10% FBS) and maintained in a cell incubator (37° C., 5% CO₂). On day 2, cells were stimulated with staurosporine (40 nM) to increase the expression of the 5-HT transporter [17]. On day 3, cells were removed from the cell incubator two hours prior to assay and maintained at room temperature to equilibrate the growth medium to ambient oxygen concentration. Subsequently, the growth medium was replaced with 200 μl of assay buffer (25 mM HEPES; 120 mM NaCl; 5 mM KCl; 2.5 mM CaCl₂; 1.2 mM MgSO₄; 2 mg/ml glucose (pH 7.4, 37° C.)) containing 0.2 mg/ml ascorbic acid and 10 μM pargyline. A stock solution of paroxetine was prepared in DMSO (10 mM) and delivered to triplicate wells containing cells for a final test concentration of 1 μM. Data from these wells were used to define non-specific 5-HT uptake (minimum 5-HT uptake). Test compound was prepared in DMSO (10 mM) and diluted in assay buffer according to test range (1 to 1,000 nM). Twenty-five microliters of assay buffer (maximum 5-HT uptake) or test compound were added directly to triplicate wells containing cells in 200 μl of assay buffer. The cells were incubated with the compound for 10 minutes (37° C.). To initiate the reaction, [³H]hydroxytryptamine creatinine sulfate diluted in assay buffer was delivered in 25 μl aliquots to each well for a final test concentration of 15 nM. The cells were incubated with the reaction mixture for 5 minutes at 37° C. The 5-HT uptake reaction was terminated by decanting the assay buffer. The cells were washed twice with 200 μl assay buffer (37° C.) to remove free radioligand. The plates were inverted and left to dry for 2 minutes, then reinverted and air-dried for an additional 10 minutes. Subsequently, the cells were lysed in 25 μl of 0.25 N NaOH (4° C.) then placed on a shaker table and shaken vigorously for 5 minutes. After cell lysis, 75 μl of scintillation cocktail was added to the wells, the plates were sealed with film tape and replaced on the shake table for a minimum of 10 minutes. The plates were counted in a Wallac Microbeta counter (PerkinElmer) to collect the raw cpm data.

Dopamine Transporter (hDAT) Membrane Binding Assay

Frozen membrane samples are diluted to 7.5 ml in binding buffer (50 mM Tris-HCl pH 7.4, 100 mM NaCl), homogenized with a tissue-tearer (Polytron PT 1200C, Kinematica AG), and delivered at a volume of 75 μl to each well of a polypropylene 96-well plate. Millipore MultiScreen-FB opaque 96-well plates (Millipore glass fiber B, Cat. No. MAFBNOB) are blocked for a minimum of two hours at room temperature with polyethylenimine (PEI; Sigma Cat. No. P-3143) diluted to 0.5% in water. The binding reaction is run in polypropylene 96-well plates (Costar General Assay Plate, Cat. No. 3359; Lid, Cat. No. 3930). Homogenized membrane prep is delivered at a volume of 75 μl to each well of a reaction plate. A stock solution of mazindol was prepared in DMSO (10 mM) and delivered to triplicate wells containing membrane for a final test concentration of 10 mM. Data from these wells were used to define non-specific (NSB) hDAT binding (minimum hDAT binding). Total binding is defined by addition of 5 μl of binding buffer alone. Test compound was prepared in DMSO (10 mM) and diluted in assay buffer according to test range (1 to 10,000 nM). Homogenized membrane are pre-incubated with test compound for 20 minutes at 4° C. before the start of the binding reaction. The binding reaction is initiated by addition of 25 μl of ³H-WIN 35,428, diluted in binding buffer, is delivered at a final concentration of 32 nM (K_{d} for Lot#2227 29.7 nM). The reaction is incubated 2 hours at 4° C. Prior to harvesting the reaction plates, the PEI block is aspirated from the filter plates using a vacuum manifold. Aliquots of each reaction (90 ∥l of each 100 μl reaction well) are transferred from the reaction plates to the filter plates using a Zymark Rapid Plate-96 automated pipette station. The binding reaction is terminated by vacuum filtration onto the blocked, glass fiber filters. The filter plates are aspirated at 5 to 10 inches Hg, and the wells are washed 9× with 200 μl ice cold wash buffer (50 mM Tris-HCl, 0.9% NaCl, pH 7.4) using a 12 channel aspirate/wash system. Plastic bottom supports are removed from the filter plates and the plates are placed in plastic holders. A 100 μl aliquot of scintillation fluid is added to each well, and the top of each plate is sealed with adhesive film. The plates are vigorously shaken for 10 to 15 minutes prior to collection of raw cpm data using a Wallac Microbeta counter (Perkin Elmer).

Evaluation of Results

For each experiment, a data stream of cpm values collected from the Wallac Microbeta counter was downloaded to a Microsoft Excel statistical application program. Calculations of IC₅₀/EC₅₀ values were made using the transformed-both-sides logistic dose response program written by Wyeth Biometrics Department. The statistical program uses mean cpm values from wells representing maximum binding (total) or uptake (assay buffer) and mean cpm values from wells representing minimum binding (NSB) or uptake ((1 μM desipramine (hNET), 1 μM paroxetine (hSERT) or 10 μM mazindol). Estimation of the IC₅₀/EC₅₀ value was completed on a log scale and the line was fit between the maximum and minimum binding or uptake values. All graphic data representation was generated by normalizing each data point to a mean percent based on the maximum and minimum binding or uptake values. The IC₅₀/EC₅₀ values reported from multiple experiments were calculated by pooling the raw data from each experiment and analyzing the pooled data as one experiment. All experiments with racemic 1-(3,4-dichlorophenyl)-3-azabicyclo[3.1.0]hexane were completed a minimum of two times in separate experiments for all assays described.

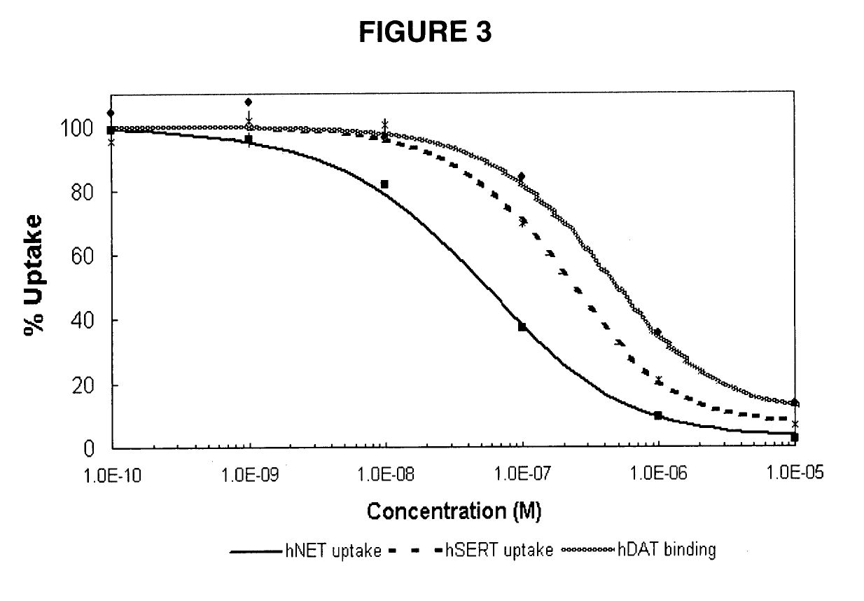

The results for racemic 1-(3,4-dichlorophenyl)-3-azabicyclo[3.1.0]hexane are shown in FIG. 3. The results for racemic 1-(3,4-dichlorophenyl)-3-azabicyclo[3.1.0]hexane, racemic 1-(4-methylphenyl)-3-azabicyclo[3.1.0]hexane and (+)-1-(4-methylphenyl)-3-azabicyclo[3.1.0]hexane are as reported below:

Racemic 1-(3,4-dichlorophenyl)-3-azabicyclo[3.1.0]hexane

| | |
|---|---|
| hNET function uptake | 51 +/− 5 |
| (EC₅₀ in nm) | 55.6 +/− 5.8 |
| | 48.6 +/− 5.7 |
| hSERT function uptake | 209 +/− 27 |
| (EC₅₀ in nm) | 215.6 +/− 39.6 |
| | 191.9 +/− 31 |
| hDAT binding | 395 +/− 111 |
| (IC₅₀ in nm) | 429.9 +/− 132.6 |
| | 374.3 +/− 71.33 |

Racemic 1-(4-methylphenyl)-3-azabicyclo[3.1.0]hexane

| | |
|---|---|
| hNET function uptake | 265 +/− 44 |
| (EC₅₀ in nm) | |
| | 426 +/− 38.0 |
| hNET binding | 71.8 |
| (% I @ 1 μM) | |
| hSERT function uptake | 736 +/− 158 |
| (EC₅₀ in nm) | |
| hSERT binding | 42.8 |
| (% I @ 1 μM) | |
| hDAT binding | 26.5 |
| (% I at 1 μM) | |
| hDAT binding | 45 |
| (% I at 10 μM) | |
| | 52.1 |

(+)-1-(4-methylphenyl)-3-azabicyclo[3.1.0]hexane

| | |
|---|---|
| hNET function uptake | 148 +/− 22 |
| (EC₅₀ in nm) | |
| hSERT function uptake | 7352 +/− 896 |
| (EC₅₀ in nm) | |
| hDAT binding | 51 |
| (% I at 10 μM) | |

### Example 2

### Telemetry Model

This model has been modified from a previously reported protocol describing estrogen regulation of diurnal tail skin temperature (TST) patterns (Berendsen, et al., European Journal of Pharmacology, 2001, 419(1): 47-54). Over a 24-hour period, intact cycling rats decrease TST during the active (dark) phase and TST remains elevated during the inactive (light) phase. In ovariectomized (OVX) rats, TST is elevated over the entire 24-hour period, thus the usual decrease in TST during the active (dark) phase is lost, thus, a compound's ability to restore this lowering of TST during the active phase was examined. A temperature and physical activity transmitter (PhysioTel TA10TA-F40, Data Sciences International) was implanted subcutaneously in the dorsal scapular region and the tip of the temperature probe was tunneled subcutaneously 2.5 cm beyond the base of the tail. After a 7-day recovery period, TST readings were continuously recorded for the remainder of the study. Tail skin temperature readings were collected from each animal every 5 minutes with values obtained over a 10 second sampling period. The day before test day, an average baseline TST value was calculated for each animal by averaging temperature readings recorded during the 12 hour active (dark) phase. In these studies, animals were dosed approximately 40 minutes prior to the onset of dark cycle.

Statistical analysis: Evaluation of a compound's ability to restore normal lowering of TST in the telemetry model was analyzed using hourly TST values calculated for each animal by averaging the 12 temperature readings obtained every 5 minutes over that recording time. To analyze ΔTST in the telemetry model, a two factors repeated measure ANOVA was performed. The model used for analysis was ΔTST=GRP (group)+HR (hours)+GRP*HR+BASELINE. Thus, the reported least squares means are the expected mean values as if both groups had the same baseline value. Post-hoc tests of hourly GRP*HR samples are t-tests of the difference between groups for each hour. To be conservative, a result was not considered significant unless the p-value was <0.025. All analyses were performed using SAS PROC MIXED (SAS, Carey, N.C.).

Rats were injected subcutaneously with vehicle (2% Tween/0.5% methylcellulose) or 30 mg/kg, sc test compound dissolved in 2% Tween/0.5% methylcellulose. The effect of test compound is measured by evaluating the following parameters in this model: onset of action, duration of effect on TST, maximal change in TST and mean change in TST over the duration of the compound effect.

Racemic 1-(3,4-dichlorophenyl)-3-azabicyclo[3.1.0]hexane, racemic 1-(4-methylphenyl)-3-azabicyclo[3.1.0]hexane and (+)-1-(4-methylphenyl)-3-azabicyclo[3.1.0]hexane and restores normal TST in an OVX-induced thermoregulatory dysfunction telemetry model (telemetry model) 30 mg/kg, sc * indicates p<0.05 compared to vehicle control.

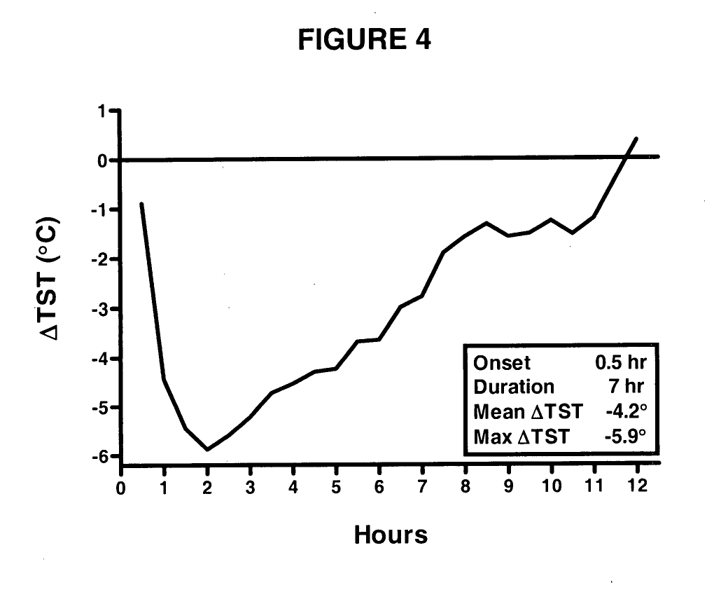

The results of the administration of racemic 1-(3,4-dichlorophenyl)-3-azabicyclo[3.1.0]hexane at 1 dose (30 mg/kg, sc) in telemetry rat model of ovariectomy-induced thermoregulatory dysfunction are shown in FIG. 4.

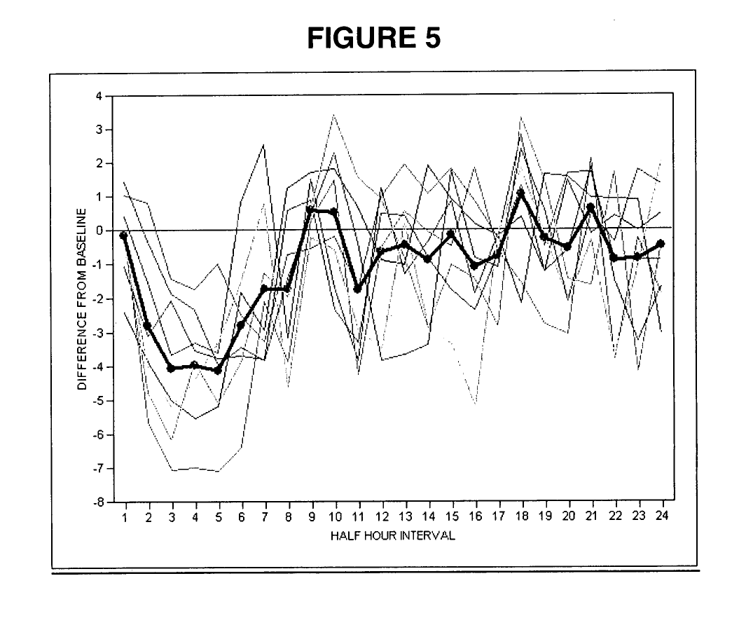

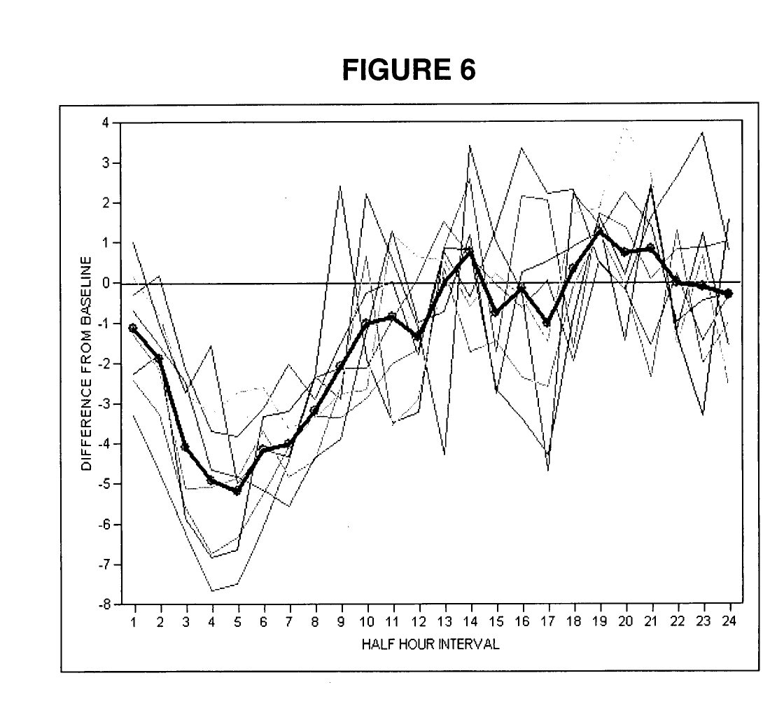

The results of the administration of racemic 1-(4-methylphenyl)-3-azabicyclo[3.1.0]hexane and (+)-1-(4-methylphenyl)-3-azabicyclo[3.1.0]hexane at 1 dose (30 mg/kg, sc) in telemetry rat model of ovariectomy-induced thermoregulatory dysfunction are shown in FIG. 5 and FIG. 6 and as shown below.

Racemic 1-(4-methylphenyl)-3-azabicyclo[3.1.0]hexane

| | |
|---|---|
| Onset (hours) | 0.5 |
| Duration (hours) | 3.5 |
| Mean (hours) | −3.02 |
| Mean Difference (hours) | −4.1 |
| Activity Index (hours) | −10.6 |

(+)-1-(4-methylphenyl)-3-azabicyclo[3.1.0]hexane

| | |
|---|---|
| Onset (hours) | 0.5 |
| Duration (hours) | 3.5 |
| Mean (hours) | −2.99 |
| Mean Difference (hours) | −3.26 |
| Activity Index (hours) | −2.99 |

### Example 3

### Evaluation of racemic 1-(3,4-dichlorophenyl)-3-azabicyclo[3.1.0]hexane, racemic 1-(4-methylphenyl)-3-azabicyclo[3.1.0]hexane (bicifadine), and (+)-1-(4-methylphenyl)-3-azabicyclo[3.1.0]hexane, in the spinal nerve ligation (SNL) model of neuropathic pain

Materials and Methods

Animal maintenance and research were conducted in accordance with the National Research Council's policies and guidelines for the handling and use of laboratory animals outlined in the Guide for the Care and Use of Laboratory Animals. The laboratory facility was licensed by the United States Department of Agriculture and accredited by the American Association for Accreditation of Laboratory Animal Care. Research protocols were approved by the Wyeth Institutional Animal Care and Use Committee in accordance with the guidelines of the Committee for Research and Ethical Issues of IASP (Zimmermann, 1983).

Subjects. Male Sprague-Dawley rats (Indianapolis, Ind.) weighing 150 to 200 g at time of arrival, were individually housed in wire cages in a climate-controlled room. A 12-hour light/dark cycle (lights on at 0630) was in effect, and food and water were available ad libitum.

Surgery—Spinal Nerve Ligation. Rats were anesthetized with 3.5% halothane in O₂ at 1 L/min and maintained with 1.5% halothane in O₂ during surgery. Ligation of the L5 and L6 nerves was produced by an incision through the left paraspinal muscles. The left L5 and L6 spinal nerves were isolated adjacent to the vertebral column and ligated tightly with 6-0 silk suture just distal to the dorsal root ganglion. The wound was closed in layers using 4-0 silk suture and wound clips. Testing began 7 days after surgery.

Assessment of tactile hypersensitivity. Animals were placed in elevated wire cages and allowed 45 to 60 minutes to acclimate to the testing room. Baseline tactile sensitivity was assessed using a series of calibrated von Frey monofilaments (Stoelting; Wood Dale, Ill.) 0 to 3 days before surgery. Von Frey monofilaments were applied to the mid-plantar hind paw in sequential ascending or descending order, as necessary, to hover as closely as possible to the threshold of responses. The threshold was indicated by the lowest force that evoked a brisk withdrawal response to the stimuli. Thus, a withdrawal response led to the presentation of the next lighter stimulus and the lack of a withdrawal response led to the presentation of the next stronger stimulus. Rats with baseline thresholds <10 g force were excluded from the study. Three to four weeks following surgery, tactile sensitivities were reassessed, and animals that failed to exhibit subsequent tactile hypersensitivity (threshold ≧5 g) were excluded from further testing. Subjects were pseudo-randomly divided into test groups of 7 so that average baseline and post-surgery sensitivities were similar among groups. The ability of a single dose of test compound to reverse established hypersensitivity was assessed using a time course procedure. Under this procedure, 30 mg/kg test compound or vehicle was administered IP and sensitivities were reassessed 30, 60, 100, 180 and 300 minutes after administration.

Results are presented as the 50% threshold values (50% threshold in g force) estimated by the Dixon non-parametric test. Fifteen-gram force was used as the maximal force. Individual tactile hypersensitivity threshold values were averaged to provide a mean response (±1 SEM). Statistical analysis was done using a one-way analysis of variance (ANOVA). Significant main effects were analyzed further by subsequent least significant difference analysis. The criterion for significant differences was p<0.05.

Reversal of tactile hypersensitivity was defined as a return to baseline of the tactile sensitivity and was calculated according to the following equation:

$$\% Reversal = \frac{\begin{matrix}( 50 \% {threshold}^{ drug + post surgery )} ) - \\ ( 50 \% {treshold}^{ post surgery )} )\end{matrix}}{\begin{matrix}( 50 \% {threshold}^{ pre surgery )} ) - \\ ( 50 \% {threshold}^{ post surgery )} )\end{matrix}} \times 100$$

in which 50% threshold^{drug+post surgery} is the 50% threshold in g force after drug in nerve injured subjects, 50% threshold^{post surgery} is the 50% threshold in g force in nerve injured subjects, and 50% threshold^{pre surgery} is the 50% threshold in g force before nerve injury. Maximal effect of 100% reversal represents a return to the mean pre-operative threshold value for subjects in that experimental condition.

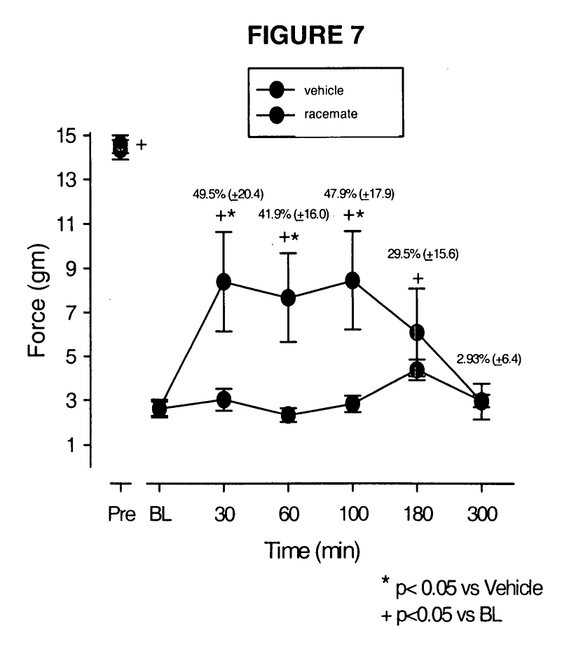

The results for racemic 1-(3,4-dichlorophenyl)-3-azabicyclo[3.1.0]hexane are shown in FIG. 7. As may be seen in FIG. 7, racemic 1-(3,4-dichlorophenyl)-3-azabicyclo[3.1.0]hexane significantly reverses tactile allodynia in the SNL neuropathic pain model. (+)-1-(3,4-Dichlorophenyl)-3-azabicyclo[3.1.0]hexane is also expected to reverse tactile allodynia in the SNL neuropathic pain model.

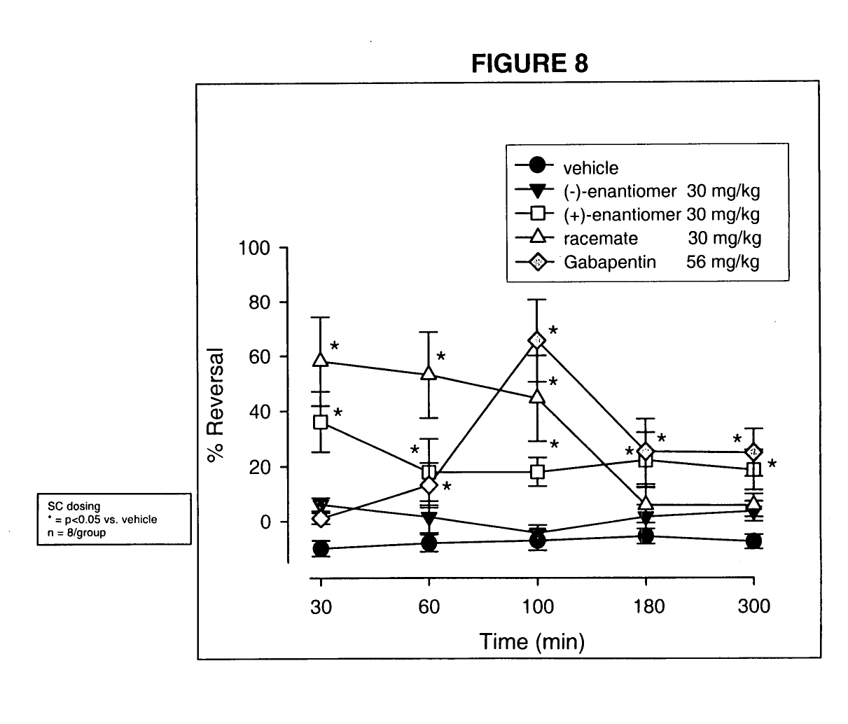

The results for racemic 1-(4-methylphenyl)-3-azabicyclo[3.1.0]hexane (bicifadine), (+)-1-(4-methylphenyl)-3-azabicyclo[3.1.0]hexane, (−)-1-(4-methylphenyl)-3-azabicyclo[3.1.0]hexane, gabapentin, and vehicle are shown in FIG. 8, which is a plot of % reversal at 30, 60, 100, 180, and 300 minutes after administration of racemic 1-(4-methylphenyl)-3-azabicyclo[3.1.0]hexane (bicifadine), (+)-1-(4-methylphenyl)-3-azabicyclo[3.1.0]hexane, (−)-1-(4-methylphenyl)-3-azabicyclo[3.1.0]hexane, gabapentin, and vehicle.

When ranges are used herein for physical properties, such as molecular weight, or chemical properties, such as chemical formulae, all combinations and subcombinations of ranges specific embodiments therein are intended to be included.

The disclosures of each patent, patent application and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method for treating at least one nervous system disorder or condition in a subject in need thereof, comprising the step of:
administering to said subject a composition comprising an effective amount of racemic 1-(3,4-dichlorophenyl)-3-azabicyclo[3.1.0]hexane, racemic 1-(4-methylphenyl)-3-azabicyclo[3.1.0]hexane, or a pharmaceutically acceptable salt thereof;
wherein said nervous system disorder or condition is a vasomotor symptom, sexual arousal and desire, fibromyalgia, chronic fatigue, hypothalamic amenorrhea, chronic pain, cognitive dysfunction associated with senile dementia, memory loss, Alzheimer's disease, amnesia, autism, Shy Drager syndrome, Raynaud's syndrome and pain associated therewith, epilepsy, Lennox syndrome, intellectual deficit associated with cerebrovascular disease, schizophrenia, schizoaffective disorder, schizophreniform disorder, seasonal affective disorder, sleep disorder, premenstrual dysphoric disorder, withdrawal syndrome, bipolar disorder, cyclothymic disorder, dysthymic disorder, generalized anxiety disorder, social phobia, selective serotonin reuptake inhibition (SSRI) poop out syndrome, panic disorder, agoraphobia, post traumatic stress disorder, borderline personality disorder, fecal incontinence, disturbances of consciousness, coma, speech disorders, or a combination thereof.

2. A method according to claim 1,
wherein said composition further comprises at least one adrenergic_{α2} receptor antagonist.

3. A method according to claim 1,
wherein said composition comprises racemic 1-(3,4-dichlorophenyl)-3-azabicyclo[3.1.0]hexane, or a pharmaceutically acceptable salt thereof.

4. A method according to claim 1,
wherein said composition comprises racemic 1-(4-methylphenyl)-3-azabicyclo[3.1.0]hexane, or a pharmaceutically acceptable salt thereof.

5. A method according to claim 1,
wherein said nervous system disorder or condition is a vasomotor symptom.

6. A method according to claim 5,
wherein said vasomotor symptom is hot flush.

7. A method according to claim 1,
wherein said subject is human.

8. A method according to claim 7,
wherein said human is a female.

9. A method according to claim 8,
wherein said female is pre-menopausal.

10. A method according to claim 8,
wherein said female is peri-menopausal.

11. A method according to claim 8,
wherein said female is post-menopausal.

12. A method according to claim 7,
wherein said human is a male.

13. A method according to claim 12,
wherein said male is naturally, chemically or surgically andropausal.

14. A method according to claim 1,
wherein said nervous system disorder or condition is chronic pain.

15. A method according to claim 14,
wherein said nervous system disorder or condition is neuropathic pain.
